(19) United States
(12) Patent Application Publication
(10) Pub. No.: US 2011/0313342 A1
(43) Pub. Date: Dec. 22, 2011

(54) Method of Treating Open Wounds and Bed Sores

(76) Inventor: Julia McCoy, Houston, TX (US)

(21) Appl. No.: 12/817,337

(22) Filed: Jun. 17, 2010

(51) Int. Cl. A61M 1/00

(52) U.S. Cl. 604/19

(57) ABSTRACT

A method for treating human pressure ulcers of bedridden patients utilizing flour. The flour is first heated over heat for a sufficient amount of time to undergo a chemical change causing the granule molecules to burst and become gelantize. Flour is a known starch and insoluble in its natural form. The browning of the flour causes a chemical reaction that allows the brown substance to more effectively absorb the moisture in the body. In this method, the brown substance is applied to the infected area each day for a sufficient amount of time until the wound heals.

### TECHNICAL FIELD

1. Background

Bed sores, more properly known as pressure ulcers or decubitus are lesions cause by many factors such as unrelieved pressure; friction; humidity; shearing forces; temperature; age; continence; and medication. The primary cure and treatment is to remove the pressure by turning the patient every two hours.

Bed sores are caused by three different tissue forces: pressure, shear force, and friction. Aggravating the situation may be other conditions such as excess moisture from incontinence, perspiration or exudates. The first and foremost accepted theory about the development of pressure ulcers is the deep tissue injury theory, which claims that the ulcers begin at the deepest level, around the bone, and move outward until they reach the epidermis.

Pressure ulcers may be caused by inadequate blood supply and resulting reperfusion injury when blood re-enters tissue. The sore will initially start as a red, painful area, which eventually turns purple. Left untreated, the skin may break open and become infected. Moist skin is more sensitive to tissue ischemia and necrosis.

Within acute care, the incidence of bed sores is 0.4% to 38%; within long-term care, 2.2% to 23%; and in home care, 0%-17%. There is a much higher rate of bed sores in intensive care units because of immuno-compromised individuals, with 8% to 40% of ICU patients developing bed sores.

The removal of necrotic tissue is an absolute must in the treatment of pressure sores. Because dead tissue is an ideal area for bacteria growth, it has the ability to greatly compromise wound healing. One way is chemical debridement or enzymatic debridement, is the use of prescribed enzymes that promote the removal of necrotic tissue. Additionally, infected wounds may have a gangrenous smell, be discoloured, and may eventually exude even more pus. In order to eliminate this bioburden, it is imperative to apply antiseptics and antimicrobials at once.

The present invention provides an efficient and effective method of treating bed sores utilizing flour.

2. Summary

A method for treating human pressure ulcers of bedridden patients utilizing flour. The flour is first heated over heat for a sufficient amount of time to undergo a chemical change causing the granule molecules to burst and become gelantize. Flour is a known starch and insoluble in its natural form. The browning of the flour causes a chemical reaction that allows the brown substance to more effectively absorb the moisture in the

### BRIEF DESCRIPTION OF THE DRAWINGS

### Detailed Description

In the embodiment of the invention, browned flour is used to treat pressure ulcers. Flour is a powder made of cereal grains or roots. It is the main ingredient of bread, which is a staple food for many civilizations. Flour contains high proportion of starches, which are complex carbohydrates also known as polysaccharides.

Polysaccharides have a general formula of Cx(H2O)y where x is usually a large number between 200 and 2500. Generally starch polysaccharides are glucose polymers in which glucopyroanose units are bonded by alpha-linkages which is made of a mixture of amylose (15-20) % and amylopectin (80-85) %. Starches are insoluble in water and are normally digested by hydrolysis catalyzed by enzymes called amylases in the human body.

In the preferred embodiment of the present invention, the starch is from wheat. Starch molecules arrange themselves in the plant in semi-crystalline granules. Each plant species have a particular starch granular size. When starch is heated it undergoes a chemical change that allows it to become soluble in water. The semi-crystalline granules structure is lost as the heat swells and bursts the granules which cause the amylase molecules to start leaching out of the granule forming a network that can hold water thereby increasing the viscosity.

Amylase or alpha-Amylase (a-Amylase) is an enzyme present in flour and saliva that breaks down starches into glucose for the purpose of digestion. For the present invention, the amylase enzyme is extracted from the flour through the use of heat.

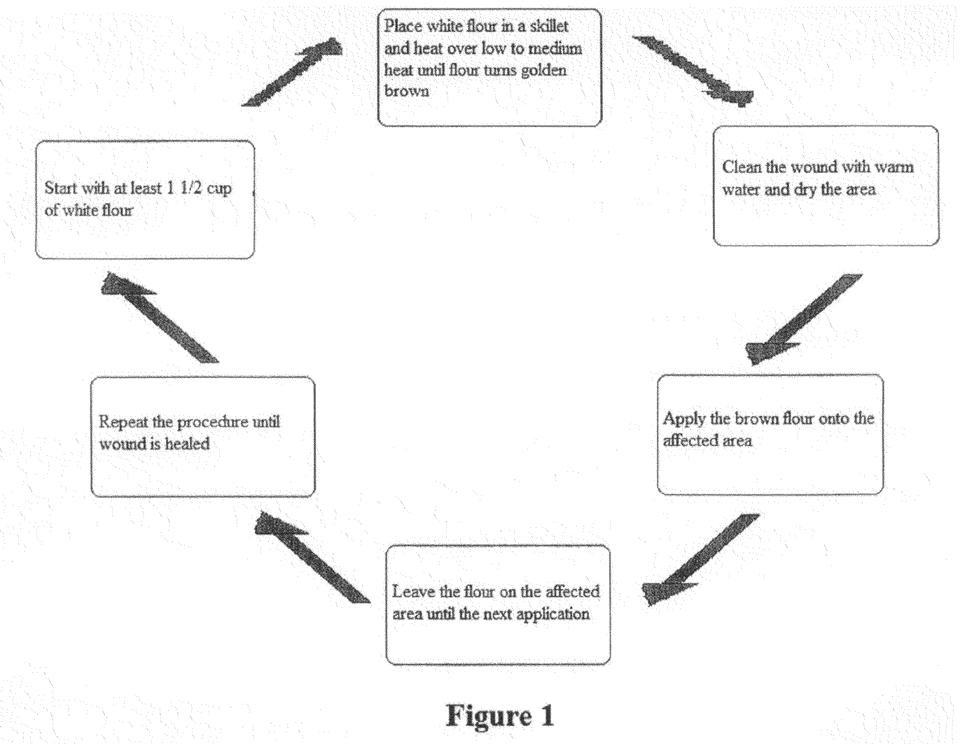

Referring to FIG. 1, there is shown an overall view of the process. The first step in the method is to release the normal enzymes stored in flour through undergoing a chemical reaction utilizing heat. Flour can first be shifted in order to remove any clumps and to give it a more powdery texture. Then the powdery textured flour is placed into a large skillet and placed over a heat source. The flour is stirred continuously over medium heat for an effective amount time until the white flour turn to a golden brown color. The effective time can be approximately between ten to thirty minutes. However, the white flour is heated until it turns to a brown color with a softer crystalline form. Then, the golden flour can be stored in a dry container.

Then, the golden brown substance is used for debridement of the dead skin. Enzymatic debridment uses enzymes to treat pressure ulcers by causing the necrotic skin to slough off. In the present invention, an effective amount of the golden flour is placed within the open sore. The golden flour amylase absorbs the moisture from the body to keep the sore dry and decrease the odor.

One of the known enzymes in the body that digests starch is amylase (a-amylase). This enzyme amalyze interacts with the leaching amylose within the browned flour thereby causing chemical or enzymatic debridement of the bed sore. The gelantized crystals of the brown substance are able to under go hydrolysis through the enzymes in the body and form a paste to protect the sore which allows the body to regenerate the skin underneath. The powder is placed on the open wound at least once a day and then wash cleaned.

The most important care for a patient with bed sores is the relief of pressure. Once a bed sore is found, pressure should immediately be lifted from the area and the patient turned at least every two hours to avoid aggravating the wound. This method in conjunction with relieving pressure can be used for treating bed ridden patients.

This method has been used on bed ridden elderly patients by the applicant as a case studies to determine. Over a period of approximately 2 to 4 weeks the sores healed. Then, the powder is used to keep the patient fresh and dry to prevent the sores from recurring.

Referring to the lists of case studies, there were 7 case studies done utilizing the product. In three of the case studies, the powder was applied to sores on three elderly women. The application of the powder healed the sores over a period of approximately two to four weeks. In two other case studies, the powder was applied to a diaper rash of young children. It was also used as a preventative measure against diaper rash two infants. Additionally the product was applied to an elderly male with remarkable results.

What is claimed is:

1. A method of preparing a composition for treating an open wound comprising:
placing an effective amount of flour in a container which can sustain a high temperature;
placing the container over heat for a predetermined cooking period of time;
continuously, stirring flour until it undergo a chemical reaction wherein a brown substance is that is water soluble is formed;
allowing the brown substance to cool;
for a predetermined treatment period of time, placing a sufficient amount of the brown substance for at least once a day upon the open wound wherein the brown substance absorbs the body fluids forming a paste over the open wound;
and repetitively treating the wound with the brown substance until the wound heals.

2. The method of claim 1 further comprises spreading a sufficient amount of the brown substance over an area of a user's body forming a thin film over the area of the user's body wherein the area of the user's body is kept dry thereby preventing the forming of a rash or a wound.

3. The method of claim 1 wherein the brown substance eliminates an odor emanating from the open wound.

4. The method of claim 1 wherein the flour is made of wheat.

5. The method of claim 1 wherein the treatment period ranges between 1 to 4 weeks.

6. The method of claim 1 wherein the temperature of the heat is medium.

7. The method of claim 1 wherein the temperature of the heat is high.

8. The method of claim 1 wherein the color of the brown substance ranges between light brown to dark brown.

9. The method of claim 1 wherein the predetermined cooking period is at least 5 minutes.
